(19) United States
(12) Patent Application Publication
(10) Pub. No.: US 2026/0345277 A1
(43) Pub. Date: Nov. 5, 2026

(54) Cobot Interface for Augmented Reality-Assisted Telesurgery Operations

(71) Applicant: Peritas AI Inc., Fort Lauderdale, FL (US)

(72) Inventor: Martin Roche, Fort Lauderdale, FL (US)

(73) Assignee: Peritas AI Inc., Fort Lauderdale, FL (US)

(21) Appl. No.: 19/428,134

(22) Filed: Dec. 20, 2025

Related U.S. Application Data
(63) Continuation-in-part of application No. 19/195,731, filed on Apr. 30, 2025.

(51) Int. Cl. A61B 34/00; A61B 34/20; A61B 34/35; A61B 90/00

(52) U.S. Cl. CPC A61B 34/76; A61B 34/20; A61B 34/35; A61B 90/03; A61B 90/361; A61B 90/39; A61B 2090/365; A61B 2090/3983

(57) ABSTRACT

A cobotic system for augmented reality-assisted telesurgery includes a robotic manipulation platform configured for precise surgical instrument handling and real-time sensor feedback. The system comprises a low-latency communication interface enabling remote control, an augmented reality display system for superimposing anatomical markers and procedural guidance on live surgical video, and a control system that synchronizes surgeon input with robotic response in real time. A multimodal sensor suite provides imaging, depth, force, and haptic feedback, while a machine learning module predicts surgeon intent to enhance responsiveness and adapt control parameters. Safety subsystems enforce sterile zone compliance, monitor for collisions, and provide emergency override protocols to ensure patient and procedural safety during remote operation.

### CROSS-REFERENCE TO RELATED APPLICATIONS

This patent application is a continuation-in-part of US Patent Application Ser. No. 19/195,731 filed on Apr. 30, 2025, which claims the benefit of priority to U.S. Provisional Patent Application No. 63/640,524, filed on Apr. 30, 2024, the entirety of which is incorporated herein by refer

### TECHNICAL FIELD

The present invention relates generally to robotic systems in surgical applications, and more specifically to cobotic interfaces configured for augmented reality-assisted telesurgery.

### BACKGROUND

Telesurgery has emerged as a powerful tool for expanding access to expert surgical care, enabling experienced surgeons to perform or assist in procedures from remote locations. By leveraging advances in robotics and communication networks, telesurgical systems allow surgical commands to be issued from a distant workstation and executed by robotic arms at the patient site. However, current telesurgery platforms remain limited in key respects, particularly with respect to immersion, responsiveness, and intraoperative adaptability.

One limitation of existing telesurgery systems is the absence of real-time, immersive feedback. Remote surgeons typically view the surgical field through fixed or limited-angle camera feeds, lacking spatial context and depth perception. Furthermore, conventional telesurgical platforms often lack augmented reality (AR) integration, preventing the overlay of anatomical landmarks, surgical plans, or dynamic procedural cues onto the operative video stream. This reduces situational awareness and increases cognitive load, particularly during complex or minimally invasive procedures.

Another shortcoming lies in the incomplete synchronization between surgeon input and cobotic response. Delays introduced by network latency, mechanical inertia, or limited sensor feedback may reduce the precision and responsiveness of instrument control. Additionally, most current systems do not dynamically adapt to intraoperative changes, such as shifts in anatomy or tool configuration, nor do they anticipate surgeon intent based on prior input patterns or surgical context.

Current platforms also lack integrated predictive intelligence that could improve workflow efficiency and safety. Although many systems incorporate basic motion filtering or stabilization, few utilize machine learning to optimize control parameters or enhance real-time responsiveness based on surgeon behavior. Similarly, while robotic arms may be equipped with basic sensors, they generally lack the multi-modal sensor fusion, including imaging, force, depth, and haptic input, needed to provide comprehensive intraoperative awareness and haptic realism to the remote operator.

Safety and regulatory compliance pose further challenges. Telesurgical systems must operate within stringent sterile boundaries, avoid collisions or unintended contact with tissues and instruments, and provide emergency intervention mechanisms in the event of hardware or communication faults. Existing systems often rely on manual overrides or human supervision to detect and respond to anomalies, limiting their capacity for autonomous fail-safe operation.

Accordingly, there is a need for an advanced telesurgical interface that integrates real-time cobotic manipulation, immersive AR visualization, multi-sensor input, and predictive intelligence into a cohesive system. In particular, a cobotic platform that supports augmented reality-guided remote operation, with synchronized feedback loops and machine-learning-enhanced responsiveness, would improve surgical precision, enable more effective collaboration between remote and local teams, and expand access to high-quality surgical care across geographic boundaries.

### SUMMARY

The present invention relates to a cobotic interface for augmented reality-assisted telesurgery operations, configured to enable real-time remote surgical intervention through integrated robotic manipulation, augmented visualization, and multimodal sensory feedback. The system includes one or more robotic arms configured to manipulate surgical instruments with high precision under the control of a remote surgeon. A low-latency communication interface enables secure, bi-directional transmission of control signals and sensor data between the surgical site and a remote operator station.

An augmented reality (AR) display system overlays live surgical video with virtual anatomical markers, surgical pathways, and procedural cues, providing the remote surgeon with enhanced spatial context and visual guidance during the procedure. The robotic arms are equipped with a multimodal sensor suite that includes imaging sensors, depth cameras, force sensors, and haptic feedback elements. These sensors generate real-time intraoperative data, which is used to inform cobot motion, update AR overlays, and generate tactile feedback for the remote surgeon.

A control system synchronizes remote operator input with cobot response in near real time through a closed-loop control and feedback architecture. The system further includes a machine learning module trained on historical and live surgeon input to anticipate surgical intent and dynamically adjust cobot responsiveness, tool orientation, and AR content. Integrated safety and compliance protocols provide collision detection, sterile field boundary enforcement, and emergency override capabilities.

The invention may be embodied as a fully integrated telesurgical system or as a modular interface compatible with existing robotic platforms, enabling remote surgical control enhanced by real-time augmented visualization, multimodal sensory feedback, and intelligent control adaptation.

### BRIEF DESCRIPTION OF DRAWINGS

The foregoing and other objects, features, and advantages of the invention will become more apparent from the following detailed description, taken in conjunction with the accompanying drawings, in which:

FIG. 1 is a perspective view of the cobotic telesurgery system.

FIG. 2 is a schematic block diagram of the system architecture.

FIG. 3 is a flow chart of the real-time control and feedback loop for synchronizing surgeon input with cobot motion and sensor output.

FIG. 4 is a process diagram illustrating the generation and delivery of augmented reality overlays based on intraoperative sensor data and surgeon inputs.

FIG. 5 is a flow chart of the machine learning interface process for safety and compliance mechanisms.

These drawings are provided for illustrative purposes only and should not be construed as limiting the invention to the specific embodiments shown and described herein. Variations and modifications to these embodiments will be apparent to those skilled in the art, and all such variations and modifications are intended to be included within the scope of this disclosure.

### DETAILED DESCRIPTION

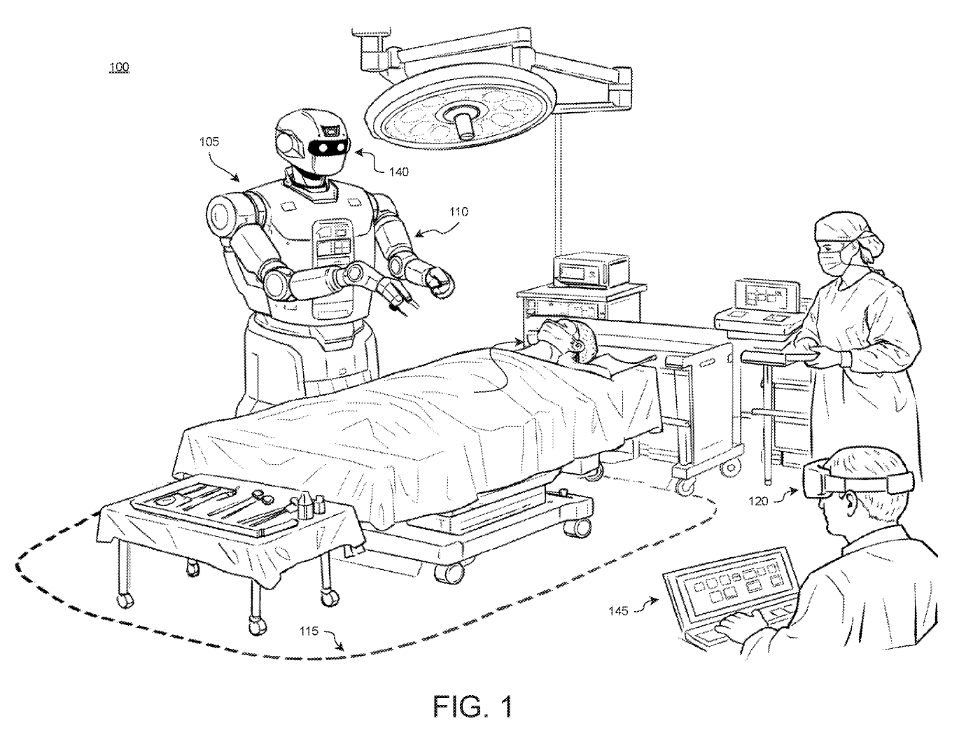

Turning to the drawings, FIG. 1 illustrates an example cobotic telesurgery system 100, showing its major components. In this embodiment, the system 100 includes a cobot 105 with a generally humanoid form factor, including a base or torso supporting one or more multi-jointed robotic arm 110. The arm 110 provides surgical manipulation capability with multiple degrees of freedom (e.g., mimicking a human shoulder, elbow, wrist, and hand). The distal end of arm 110 may include an end-effector or “hand” equipped with dexterous manipulators (grippers or fingers) that incorporate various sensors. For instance, the fingers can house tactile and force sensors to detect touch, pressure, or texture, as well as temperature sensors and other contact sensors for haptic feedback. These sensors form part of an integrated sensor suite 140, which further includes optical imaging devices (e.g., cameras or depth sensors) mounted in strategic locations on the cobot (such as in a head unit or on the arm itself) to capture stereoscopic views of the surgical field. In the depicted embodiment, a head or upper section of the cobot carries the integrated sensor suite 140, including stereoscopic cameras and microphones, enabling the system 100 to perceive its environment in real-time. The cobot 105 is preferably covered in a smooth, sterilizable outer shell (e.g., a removable drape or coating) so that it can be gowned and gloved for entry into a sterile surgical field. The outer shell and joints are designed to withstand sterilization processes, allowing the cobot to operate alongside human surgeons in an operating room without contamination.

Still referring to FIG. 1, the system 100 also includes an Augmented Reality (AR) display system 120 configured to provide dynamic guidance overlays to the human operators. In one arrangement, the AR display 120 may comprise a head-mounted display or a transparent AR visor worn by a surgeon or technician, through which live video of the surgical field (as seen by the cobot’s cameras) is presented with digital overlays. These overlays can include, for example, highlighted anatomical landmarks, recommended incision lines, or real-time instrument positioning guides. In other implementations, the AR display 120 may be a heads-up display or monitor that superimposes graphical guidance onto the video feed from the cobot. The AR display system 120 is communicatively linked to the cobot’s control system so that it can show overlays that move in synchronicity with the robot’s actions and the patient’s anatomy. For instance, as the cobot’s arm 110 moves or as the patient is repositioned, the AR overlay (such as a virtual guideline or target region) remains correctly registered in the surgeon’s view. This dynamic AR guidance enhances the teleoperator’s situational awareness and precision during remote surgery. In various embodiments, the AR interface is bidirectional, i.e. not only does it display information to the user, but it can also accept inputs (e.g., a surgeon’s gaze, gestures, or voice commands) via an input device 145, and those inputs are relayed back to the cobot 105. In the depicted embodiment the surgeon engages with an input device 145 in the form of a control panel configured to direct the cobot 105. In another embodiment, the surgeon might use an input device 145 in the form of an AR pointer or hand gesture to designate a point in the visual field, and the system 100 can interpret this input to adjust the cobot’s position or highlight an area of interest.

Several other subsystems are also housed within or on the cobot 105. A communication interface 130 is provided to enable low-latency, high-bandwidth data exchange between the local robotic system and a remote control console or network. This interface 130 can include wireless transceivers (for Wi-Fi, 5G, or dedicated telemedicine networks) and/or wired network connections. It ensures that control commands from a remote surgeon and sensor feedback from the cobot 105 are transmitted with minimal delay. In the illustrated embodiment, communication module 130 may be integrated into the cobot’s torso (e.g., with an external antenna), maintaining an encrypted data link to remote operators. The control system 150, which may include one or more processors, controllers, and real-time operating hardware, resides on the cobot 105 (e.g., in the torso or base) and is responsible for coordinating all movements and responses of the robotic arm 110 and other components. The control system 150 drives actuators in the joints of arm 110 according to remote commands and local autonomous algorithms, while simultaneously handling incoming sensor data. In one embodiment, control system 150 is a multi-layer controller with a high-level supervisory control (possibly running on a remote computer) and a low-level local motor controller for fine motion, both working in concert to achieve smooth and accurate teleoperation.

To further enhance performance, the cobotic system 100 incorporates a dedicated machine learning (ML) module 160, which may be implemented as software algorithms running on the control system 150 or on a connected computing device. ML module 160 processes the rich data from sensor suite 140 and from operator command patterns to predict operator intent and adapt the control parameters in real time. For example, if the remote surgeon tends to make certain repetitive movements or if a specific sequence of instrument use is common for a given procedure, the ML module 160 can learn from those patterns and, in an embodiment, eventually operate autonomously. Over time, it will begin to anticipate the surgeon’s next action or needed instrument and can pre-adjust the system (such as pre-positioning the arm or highlighting the next tool in the AR display) to streamline the workflow. The ML module 160 may employ computer vision techniques to recognize surgical instruments and anatomical features in the camera feed, and neural network models to correlate sensor inputs with likely surgical intentions. It is noted that the ML module 160 can operate initially in a recommendation or assist mode, offering suggested actions or gentle corrections, and can gradually take on more automation as confidence in its predictions grows (with the surgeon always able to override, maintaining ultimate control).

Finally, FIG. 1 also depicts safety and compliance subsystems 170, which are integrated components devoted to ensuring safe operation in the delicate surgical environment. The safety subsystem 170 comprises both hardware and software safety features. Hardware safety might include soft padding or force limiters on the robotic arm 110, as well as physical emergency stop buttons. Software safety features include real-time monitoring of forces and torques at the arm’s joints (via the sensor suite 140) to detect any abnormal resistance or potential collision with patient or staff. For instance, if the arm 110 or its surgical tool encounters an unexpected obstacle or excessive force, the safety controller 170 can automatically halt or retract the arm to prevent injury. Another aspect of subsystem 170 is sterile zone 115 enforcement. The system 100 may define virtual boundaries corresponding to the sterile field 115 around the patient. Position sensors and vision systems track the cobot’s limbs relative to these boundaries, and safety subsystem 170 will constrain or slow movements that approach the edges of the sterile zone 115, thereby preventing the robot from breaking sterility (for example, by avoiding contact with non-sterile surfaces or by alerting if a component nears an unsterile area). The safety subsystem 170 also monitors the status of communication link 130; if a network latency spike or drop-out occurs that could compromise control, it can momentarily pause the robot’s motion or switch to a safe autonomous mode until stable teleoperation is restored, as described further below.

Although FIG. 1 illustrates a humanoid-form cobot 105 with two robotic arms 110 and a head-mounted sensor cluster, it will be appreciated that the system 100 can assume various other physical configurations without departing from the scope of the invention. In some embodiments, the cobot 105 may include multiple robotic arms 110, e.g., a pair of arms akin to a human’s left and right arms, to perform bimanual surgical tasks. In other embodiments, the robot need not resemble a humanoid: for example, the system could be implemented as a robotic surgical cart or an overhead-mounted surgical manipulator. In a non-humanoid variant, one or more robotic arms 110 might be attached to a ceiling-mounted gantry or a bedside column, each arm still incorporating the same sensor suite 140 and controlled by the same core systems (120, 130, 150, 160, 170). The modularity of the system allows different tool attachments and sensor packages to be swapped in as needed. For instance, the distal end of arm 110 can be configured with quick-change tool mounts so that different surgical instruments or imaging devices can be attached for different procedures. One procedure might require an ultrasound probe or an endoscopic camera at the arm’s tip, while another might attach a needle driver, an electrocautery instrument, or even a radiographic imaging device. The system 100 flexibly supports such alternate configurations, and all such variants share the integrated control, AR, communication, ML, and safety features described herein. It should therefore be understood that references to a “humanoid” cobot or specific components are for illustrative purposes, and the invention encompasses numerous cobotic forms, whether anthropomorphic or not, and whether mobile or fixed in structure.

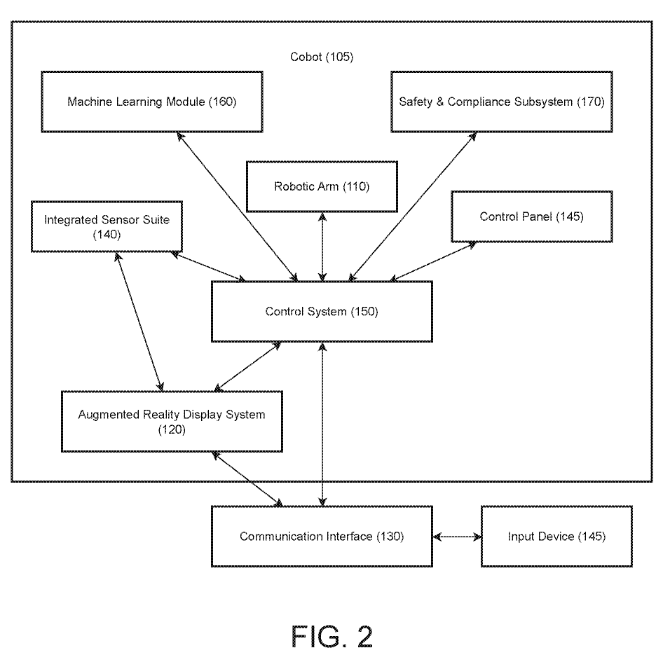

FIG. 2 provides a high-level block diagram of the system 100, showing how the various subsystems interconnect and communicate. In the diagram, the robotic arm 110 (and any associated end effector tools) is controlled by the central control system 150, which may include one or more motor controllers, processors, and control algorithms. The sensor suite 140 (including imaging devices, depth sensors, force/torque sensors, haptic feedback sensors, etc.) is distributed on the robotic arm 110 and other parts of the cobot 105, and all sensor outputs feed into the control system 150 for processing. For example, high-resolution video from cameras in the sensor suite 140 is transmitted to both the control system 150 and the AR display system 120, while force sensor data (from the arm’s joints or gripper) is sent to the control system 150 to facilitate force feedback control loops.

As shown in FIG. 2, the AR display subsystem 120 interfaces with the control system 150 and the communication interface 130. In one embodiment, AR display 120 receives a video feed and telemetry data from control system 150 (for instance, the position of arm 110 and identification of objects in view) and overlays graphical guidance elements onto the video in real time. The AR display 120 may reside at a remote surgeon’s console or on a local user’s headgear, but functionally it is part of the overall system’s feedback pathway. The communication interface 130 is depicted linking the local cobot’s control system 150 with a remote operator console (not numbered in the figure). This bidirectional link carries control commands (e.g., motion commands, tool actuation signals, or camera orientation instructions) from the remote user to the control system 150, and carries feedback data (video streams, haptic feedback signals, system status) from the cobot 105 back to the remote user. The communication interface 130 can include multiple channels or protocols – for example, a high-priority real-time channel for control signals and haptics, and a separate high-throughput channel for video and audio streaming. In some embodiments, interface 130 employs a combination of wired links (such as fiber optic lines for minimal latency) and wireless links (for mobility), using whichever channel ensures the lowest latency and highest reliability at any given moment.

Within control system 150, the machine learning module 160 is shown connected to both the sensor suite 140 and the control logic. The ML module 160 continuously analyzes incoming data (such as the video feed, the positions/velocities of the robotic arm, and even the surgeon’s command inputs over time) to extract patterns and inform the control strategy. For instance, module 160 might run a trained neural network that detects surgical context from camera images – distinguishing, say, a suturing phase versus a tissue resection phase – and proactively adjusts the system’s behavior (different control sensitivity or suggested tools) accordingly. The ML module 160 also interfaces with the AR display 120, in that it can trigger certain visual overlays or alerts. By way of example, if the ML predicts that a surgeon is about to operate on a particular anatomical structure (based on the context and prior learned data), it might command the AR system 120 to highlight that structure or display a reminder of a checklist relevant to that step.

The safety and compliance subsystem 170 is also illustrated in FIG. 2 as an oversight layer interfacing with the control system 150, the sensor suite 140, and the actuators of the robotic arm 110. Safety subsystem 170 receives raw and processed sensor data (like distances to obstacles from depth sensors, or force readings indicating contact) and can directly intervene in the control signals. In normal operation, control system 150 sends motor commands to the arm 110 to execute the surgeon’s inputs, but the safety subsystem 170 can modify or veto these commands in milliseconds if a hazard is detected. For example, if a nurse or another surgeon unexpectedly reaches into the operating field and the sensor suite 140 detects this, subsystem 170 can limit the arm speed or stop movement to avoid even a minor collision. Likewise, if the arm 110 is about to move beyond a predefined safe region (e.g., outside the sterile field or into an area not covered by imaging), the safety logic can constrain that motion. All subsystems are thus operatively connected: the control system 150 orchestrates normal operation, ML module 160 optimizes and predicts actions, AR display 120 communicates information to the human operators, communication interface 130 links the remote and local ends, and safety module 170 provides a continuously running check on system behavior.

Preferably, the architecture is designed with redundancies and fail-safes. FIG. 2 shows, for instance, that both the control system 150 and safety subsystem 170 have access to sensor data independently – the safety subsystem 170 may be implemented on a separate hardware thread or processor that can take control if the primary controller fails or is too slow. The communication interface 130 may also include redundant links (multiple network connections) to guarantee command and data pathways remain intact. Data logs from all components (e.g., video recordings, sensor readings, command histories) can be stored locally or in a secure cloud database for later analysis, training improvements, or medical record keeping. In sum, FIG. 2 depicts an integrated system architecture in which all modules work in concert to enable augmented reality-assisted telesurgery: the robotic arm 110 and sensor suite 140 physically interact with the surgical environment, the control system 150 and ML module 160 provide intelligent command and coordination, the AR display 120 and communication interface 130 connect the human surgeons to the remote scene, and the safety subsystem 170 underpins the entire operation with real-time protective measures.

Taken together, FIGS. 1 and 2 illustrate an example deployment scenario for the system 100, highlighting the interaction between a remote surgeon (or other operator) and the local cobot 105 in the operating room. In this figure, the cobot 105 is positioned adjacent to an operating table with a patient (not explicitly numbered), and a remote surgeon at a console is controlling the cobot’s actions via the low-latency communication link 130. The remote surgeon’s console is equipped with input devices (for example, joysticks, haptic gloves, or a master robotic controller that the surgeon manipulates) and an AR display 120. Through the AR display system 120, the surgeon sees a live, real-time feed of the surgical field from the cobot’s point of view, augmented with computer-generated overlays. These overlays might include visual indicators such as a superimposed path for an incision, labels identifying anatomical structures, or markers showing where to position an instrument. The augmented reality feed allows the remote surgeon to maintain keen situational awareness despite not being physically present. In some embodiments, the AR display is realized with a stereoscopic head-mounted display, giving the surgeon a 3D view of the patient and the cobot’s arm 110 in relation to the patient, with depth cues and annotations appearing at the correct spatial locations.

The communication interface 130 is engineered for ultra-low latency, as even small delays in a telesurgery context can affect surgical precision. The system leverages high-speed data transmission (e.g., fiber optics or 5G networks) and optimizes data streams to minimize lag. For instance, the video feed sent to the remote AR display 120 can be adaptively tuned: if network conditions worsen, the system may temporarily reduce frame rate or resolution to ensure the control commands and haptic feedback remain in near-real-time sync. This adaptive streaming approach maintains the fidelity of critical visual information while keeping latency within safe bounds. In practice, latencies on the order of only a few tens of milliseconds can be achieved, which is nearly imperceptible to the operator and allows fine motor control to be exercised confidently from afar. The communication subsystem 130 also ensures that haptic feedback (force sensations) from the cobot’s sensors is relayed to the remote surgeon’s input devices without noticeable delay, so that the surgeon can “feel” the tissues or resistance as if they were operating locally. For example, when the cobot’s arm 110 makes contact with an organ or when it encounters a different tissue density while cutting, the force sensors in sensor suite 140 detect this and the control system 150 sends a corresponding haptic signal back to the surgeon’s console. The surgeon’s haptic hand controller will generate an equivalent force or vibration, thereby closing the sensory loop.

The telepresence capabilities of system 100 are not limited to a single surgeon. Because the system streams rich data (video, audio, sensor data) through interface 130, multiple remote participants can join or observe the session in real time. For instance, a remote surgical assistant or specialist could participate through their own AR display, seeing the same augmented view and even annotating or pointing out features in the shared AR space. The system 100 supports such multi-user collaboration by broadcasting the feed to authorized remote clients, each potentially with their own AR visualization. In the illustrated context, one can imagine an expert consultant or a medical device representative virtually “over the shoulder” of the operating surgeon: the consultant might draw a circle in their AR view to highlight an anatomical region, and that annotation would appear in the surgeon’s AR display as well as possibly on a local heads-up display for the OR staff. The communication system 130 and AR system 120 together handle these collaborative overlays, enabling what is effectively an interactive, global tele-surgery environment.

From the perspective of the local operating room (OR), FIG. 1 shows that the cobot 105 stands in for a human presence. The cobotic arm 110 can perform tasks such as holding instruments, retracting tissue, making incisions, or suturing, under the control of the remote surgeon. In some embodiments, the cobot 105 also has an anthropomorphic appearance with a “head” that contains cameras and possibly a screen or lights indicating its status. OR personnel (e.g., nurses or technicians physically present) may also wear AR glasses or have a display that lets them see what the remote surgeon is seeing. In this way, everyone shares a common view augmented with the same guidance cues. For example, if the remote surgeon draws a line in AR indicating where a tool on the back table is located or which instrument to pass next, the local nurse can see that line through their AR glasses, streamlining communication. The system thus creates a unified mixed-reality workspace linking remote and local participants.

Communication interface 130 additionally supports audio and voice communication as part of the telesurgery session. FIG. 1 implies that the remote surgeon and the OR team are in constant contact (e.g., via a live audio feed and possibly a video feed of the surgeon). Voice commands from the surgeon can be recognized by the system as control inputs as well – for instance, the surgeon might say “increase magnification” or “next instrument: forceps,” and the system (via control module 150 and ML module 160) will respond by zooming the camera or prompting the OR technician for the specified instrument. The ML module 160 can assist by filtering and interpreting these voice commands in context, distinguishing them from casual dialogue. As disclosed in the Aggregated Disclosure, the system can even use verbal communication followed by predictive automated tasks, meaning the surgeon can verbally instruct the cobot 105 and the system will carry out the command and predict subsequent steps. Overall, FIGS. 1 and 2 illustrate that in deployment, the system 100 enables a remote surgeon to perform surgery with local collaboration as if present, using augmented reality overlays for guidance and a robust low-latency link for telepresence and control. This configuration greatly expands access to expert surgical care by allowing skilled surgeons to operate across distances, and it enhances the precision and safety of such operations through the integrated AR and sensor feedback.

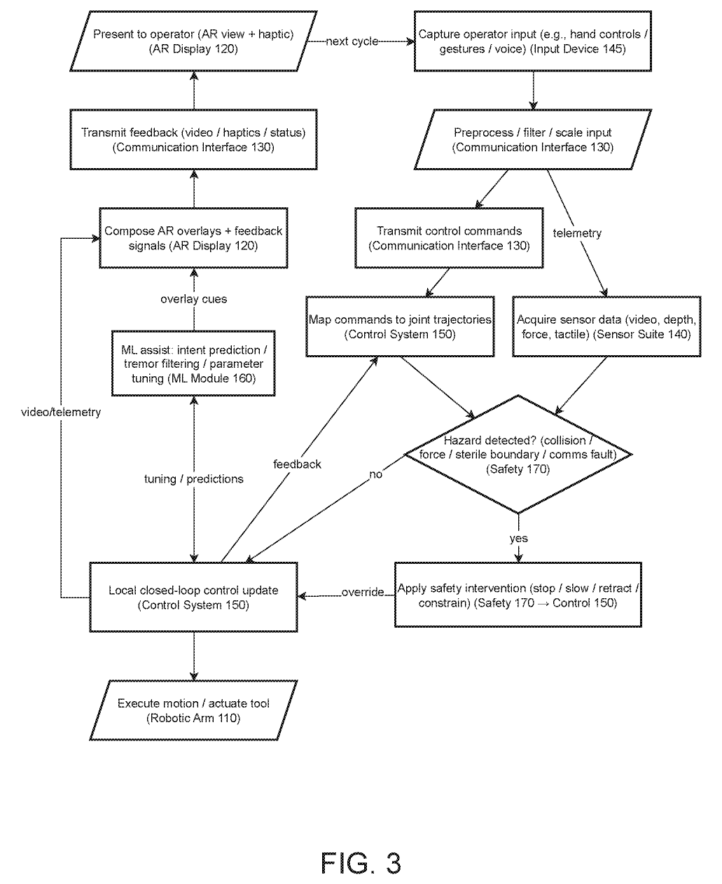

FIG. 3 is a schematic representation of the control and feedback loops in the cobotic telesurgery system, detailing how user commands, system actions, and sensor feedback are synchronized in real time. At a high level, FIG. 3 shows two primary loops: (1) the forward control loop from the remote operator to the robot, and (2) the feedback loop from the robot’s sensors back to the operator. These loops are tightly integrated to achieve a smooth, near-real-time interactive experience despite the physical separation.

In the forward control loop (as diagrammed in FIG. 3), the remote surgeon’s input device (illustrated on the left) captures the surgeon’s control actions, e.g. motions of a master controller that correspond to desired movements of the robotic arm 110. These user inputs (which can include translational/rotational motions, grip commands, tool activation, etc.) are first processed by the remote interface software (which can be considered part of or working with communication module 130). The inputs are then transmitted via the communication interface 130 to the local control system 150. The control system 150 maps the incoming commands to the cobot’s actuators: for instance, if the surgeon moves their controller 2 cm to the right, the control system 150 computes corresponding joint trajectories for the robotic arm 110 to achieve an equivalent 2 cm movement in the patient’s body frame. High-frequency servo control is executed by control system 150 to drive the robotic arm’s motors in a precise and coordinated fashion, reproducing the surgeon’s hand movements at the surgical site. Because of the critical nature of these motions, the control loop prioritizes real-time performance. As noted earlier, the communication interface 130 and control system 150 employ strategies like data compression and predictive signal smoothing to compensate for any network latency or jitter. In some embodiments, the control system 150 utilizes prediction algorithms (part of ML module 160) to estimate the surgeon’s next micro-movement fractionally ahead of time, allowing the robot to initiate that movement slightly in advance and thus cancel out transmission delay. This form of feed-forward control can be based on modeling the surgeon’s hand dynamics or using an AI that has learned typical hand motion patterns in surgery. As a result, even if the network introduces, say, 50 milliseconds of delay, the predictive control can make the robot’s motion appear perfectly synchronized with the surgeon’s actions, greatly enhancing the teleoperative experience.

Concurrently, the feedback loop, as diagrammed in FIG. 3, ensures the surgeon receives immediate sensory information from the remote site. The sensor suite 140 continuously gathers data, e.g. live video of the surgical field, force/torque measurements at the instrument tip, tactile sensor readings, audio from the patient’s environment, etc. This raw sensor data is streamed into both the control system 150 and the communication interface 130. The control system 150 uses the data for local decision-making (for example, maintaining a steady force on tissue by adjusting motor current based on force sensor input). Simultaneously, the communication interface 130 packages the sensor data (after any necessary processing, like compressing video frames or encoding haptic signals) and transmits it back to the remote surgeon’s console. There, it drives the AR display 120 and the surgeon’s haptic feedback devices. FIG. 3 shows that the video feed from the cobot’s cameras is sent to the AR display 120 where graphical overlays (from the ML module 160 or pre-operative planning data) are superimposed before presentation to the surgeon. The force feedback is routed to haptic actuators in the surgeon’s control handles so the surgeon feels forces corresponding to interactions at the remote site. This closed-loop feedback is calibrated such that, for example, if the cobot’s end effector touches a hard object in the patient, the surgeon feels a proportional resistance in their controller, and if the robot’s camera sees a particular landmark, a highlighting overlay might appear on the surgeon’s screen to draw attention to it.

FIG. 3 illustrates how the control system 150 and ML module 160 are interwoven in both the forward and feedback paths. The ML module 160 can filter and interpret sensor data to provide higher-level feedback to the surgeon. For instance, rather than just raw video, the system (via computer vision) might identify a blood vessel and outline it in the AR feed; rather than just a raw force value, the system might infer “tool is slipping” and provide a specific haptic pulse to warn the surgeon. On the forward side, ML module 160 might detect an anomalous pattern in the surgeon’s input (e.g., a tremor or an unintended movement) and automatically apply damping or error correction through control system 150 to avoid any accidental robot motion. This cooperative arrangement means the system is continuously assisting and enhancing the surgeon’s control, akin to a smart power steering in a car, where the human is always in command but subtle adjustments by the machine improve precision and safety.

FIG. 3 shows feedback to the safety subsystem 170 as well. At every control cycle, the safety monitors are checking positions, speeds, and forces. If any parameter goes out of the safe range (for example, the arm 110 moves faster than a threshold or a force exceeds a safe limit on tissue), safety subsystem 170 can intervene by modulating the control loop. It might reduce the gain on the control input (making the robot move more slowly than the surgeon’s sudden movement if it deems that movement too abrupt for safety), or insert a virtual fixture that the robot’s arm will not cross (even if the surgeon’s hand moves further in that direction). The surgeon may feel this as a gentle resistance or boundary in their haptic control, indicating a restricted action. This mechanism ensures that even under remote operation, the system maintains active constraints to protect the patient and the surgical team.

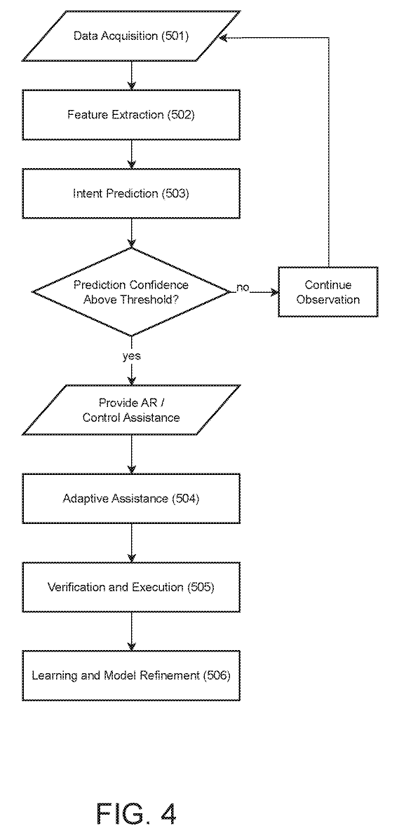

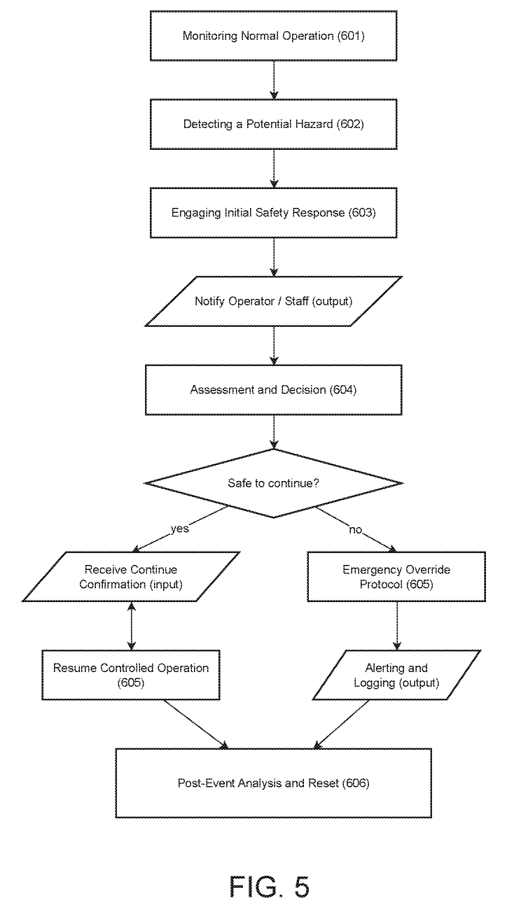

In summary, FIG. 3 demonstrates the robust control architecture enabling the cobotic system to function as an extension of the surgeon. By maintaining a tight synchronization between user commands and robotic response, and by delivering rich, real-time feedback (visual, haptic, auditory) to the operator, the system achieves a true telepresence effect. The operator can act with confidence as if physically present, while the system’s underlying intelligence (control algorithms, ML predictions) and safety checks run transparently in the background. The result is a controlled, low-latency feedback loop that merges human skill with robotic precision. Notably, the design allows for scalability and network adaptability: for instance, the system can accommodate multiple remote viewers as noted (streaming to each with potentially optimized quality per their network), and can fail gracefully (if connectivity drops, the robot can either pause or continue a pre-programmed safe action until control is restored). This architecture is foundational to supporting advanced features described in subsequent figures, such as predictive automation (FIG. 4) and safety protocols (FIG. 5).

FIG. 4 depicts an example process flow for the machine-learning-based intent prediction and adaptive control functionality (primarily handled by ML module 160 in coordination with control system 150). This process enables the cobotic system to learn from data and improve its assistance over time, enabling automation and ultimately enhancing surgical efficiency and precision. The steps of this process, which may cycle continuously during operation, can be described as follows:

Data Acquisition (Step 501): The system continuously captures multimodal data from the ongoing procedure. This includes the surgeon’s control inputs (motion commands, tool selections, etc.), sensor suite 140 readings (video images, depth maps, force feedback, etc.), and contextual information (the type of procedure, patient data, etc.). All these data streams are time-synchronized and logged. For example, as the surgeon performs a suturing motion, the system records the exact hand movements, the resulting robot arm trajectory, the visual feed of the needle and tissue, and the force applied. Over many such instances, a rich dataset is accumulated.

Feature Extraction (Step 502): The ML module 160 processes the raw data to extract meaningful features that characterize the surgical context and the operator’s intent. Computer vision algorithms might identify objects in the camera feed (such as surgical instruments or anatomical structures) and track their positions. From the control input patterns, the system can derive features like movement speed profiles, tremor frequency, or common sequences of actions. It may also parse the surgeon’s verbal commands or gestures if those are part of the input. For instance, the system notes that whenever the surgeon says “next,” it is followed by a specific instrument change. These features are fed into predictive models.

Intent Prediction (Step 503): Using machine learning models (e.g., trained neural networks or probabilistic models), the system predicts the operator’s intent or next action. Based on the current state and recent patterns, the ML module 160 generates a hypothesis of what the surgeon likely wants to do next. For example, if the surgeon has been tying a suture and is nearing completion, the model might predict that the surgeon will next want the scissors to cut the suture. Similarly, if a certain anatomical landmark comes into view, the system might predict that a measurement or cauterization step is coming up. The prediction might be in the form of a ranked list of likely next actions or tool uses, with confidence scores.

Adaptive Response/Assistance (Step 504): The system then adapts the control scheme or interface in anticipation of the predicted intent. Several adaptive strategies can occur at once:

Pre-positioning or Aligning: Control system 150 can start subtly adjusting the robotic arm 110 or camera angle to be optimally positioned for the predicted next maneuver. For instance, if a certain angle or zoom is usually used for the next step, the camera might begin moving to that position in advance.

Parameter Tuning: The system can modify control sensitivity or constraints. If a delicate maneuver is expected, it might temporarily reduce the gain on the surgeon’s inputs (making the robot movements finer and more tremor-filtered) to improve precision.

Cueing the Operator: Through AR display 120, the system can present visual or audio cues. For example, highlighting the predicted instrument on the AR overlay (or even on a connected instrument tray system) to prompt a human assistant to get it ready. The AR system might also outline the region of interest on the patient where the next action is likely, focusing the surgeon’s attention.

Automation of Subtasks: In some cases, if confidence is high and if the system is authorized, it might carry out a micro-action autonomously. For instance, the ML module might trigger the cobot 105 to begin opening an instrument clamp or aligning a needle at a certain approach angle, so that when the surgeon goes to perform the action, it is already partially done (reducing effort).

This adaptive assistance is done carefully to always remain non-obtrusive and under surgeon control. The surgeon can always ignore or override suggestions. The system essentially acts as a highly intelligent surgical assistant, anticipating needs and smoothing the workflow.

Verification and Execution (Step 505): As the predicted action window arrives, the system monitors the surgeon’s actual input and the outcome. If the prediction was correct (say the surgeon indeed requests or moves toward the anticipated action), the preemptive adaptations simply make that action more seamless — the tool is ready, the view is perfect, etc., so the surgeon experiences less friction. If the prediction was wrong, the system either reverts any preparatory adjustments or smoothly adjusts to the actual intent. The control system 150 always ensures that any autonomous moves made in anticipation do not conflict with the surgeon’s real-time commands. At this step, the intended surgical action is executed with the combined effort of the surgeon and the adaptive control system. For example, the surgeon’s command to move to a location is executed, perhaps faster or more accurately than it would have been without assistance, because the system had already begun the motion.

Learning and Model Refinement (Step 506): After execution, the system updates its machine learning models based on what actually happened. If the prediction was off, the model learns from that mistake to improve future accuracy. If the prediction was correct, that positive reinforcement strengthens the model’s confidence in similar situations. The data from this operation (features, predictions, outcomes) are added to the training dataset. Over time, with many surgeries and perhaps many different surgeons, the ML module 160 becomes more adept. It can even personalize its behavior to a particular surgeon’s style. For instance, one surgeon might like a certain instrument earlier than others during a procedure – the system will learn and adapt to these individual preferences. Additionally, aggregated data from multiple procedures allow the system to refine general surgical workflow models.

By iterating through these steps continuously, the cobotic system transitions from a purely reactive machine to a proactively assistive partner in surgery. FIG. 4’s process can run in parallel with direct teleoperation, meaning the surgeon does not have to wait for any of these steps because they are happening in the background, milliseconds to seconds ahead of the surgeon’s actions. The outcome is a reduction in cognitive load and an increase in efficiency. For example, machine learning-driven assistance might shave minutes off procedure time by ensuring that the next tool is always ready or by preventing missteps (like an accidental excessive force that could cause bleeding, which the system might avert by modulating input as described). In one embodiment disclosed in the supporting materials, a surgeon can input a specific procedure profile or preference set at the start, and the cobot 105 will configure a series of pre-set steps and recognitions for predictive assistance. For instance, in an orthopedic surgery, if the surgeon indicates they will perform a knee replacement, the system can load a model trained on knee replacement workflows. The ML module 160 then knows typical sequences (e.g., remove damaged tissue → drill femur → place implant), common points where the surgeon might need help (like aligning cutting guides), and patient-specific data (imaging scans that can overlay via AR for alignment). Throughout the operation, it provides a tailored assist: graphically communicating via AR, responding to voice commands or hand gestures, and even partially automating routine sub-tasks. All the while, it keeps the surgeon in the loop and allows manual override for any automated suggestion – a design principle that ensures safety and control remain paramount even as automation increases.

Notably, as the system gathers more experience, it can progress from semi-autonomous assistance to handling certain routine tasks autonomously under supervision. For example, it might suture a closing incision on its own once the surgeon gives a command, because it has learned that task thoroughly. However, the preferred implementation is to always have a human in control or supervising, with the ML-based automation serving to augment and expedite, not to completely take over (except in well-defined scenarios like emergency fail-safes). In short, FIG. 4 shows the pathway by which the inventive system leverages machine learning and artificial intelligence to continuously improve its performance – enabling predictively accurate, personalized, and adaptive control in telesurgery. This intelligence layer builds upon the core teleoperation capabilities of the system (described earlier) and sets the stage for a new era of surgical robotics where the machine and surgeon work in true synergy.

FIG. 5 illustrates a representative workflow 600 for the system’s safety and compliance mechanisms, including how safety protocols are engaged and how an emergency override is handled. This flowchart details the steps the system 100 takes to prevent or respond to unsafe conditions during teleoperated surgery, thereby protecting the patient and surgical staff at all times. The process can be summarized in a series of stages:

Monitoring Normal Operation (Step 601): In real-time during surgery, the safety subsystem 170 continuously monitors sensor data and system status. This includes tracking the positions and velocities of the robotic arm 110, the proximity of the arm (and any attached instruments) to the patient and to defined no-go zones, the forces being applied to tissues (from force sensors in sensor suite 140), and the status of the communication link 130 (latency, packet loss, etc.). The system effectively establishes a baseline of safe operation parameters – for example, “arm moving within allowed region, force below threshold X, network latency < Y ms.” As long as all readings are within predetermined safe limits, the surgery proceeds under normal teleoperative control.

Detecting a Potential Hazard (Step 602): If any monitored parameter deviates from the safe envelope, the safety subsystem 170 detects a potential hazard condition. Hazards can take many forms, such as:

Obstacle or Collision Risk: The vision sensors or proximity sensors see an object (e.g., a human hand or an unexpected instrument) entering the cobot’s workspace, or the arm 110 gets too close to an obstacle. For instance, if a nurse leans in, the cobot’s optical sensors detect the person’s presence.

Excessive Force or Strain: A force sensor on the arm or gripper registers a force above the allowed threshold (indicating the cobot might be pressing too hard on tissue or stuck against a rigid structure).

Sterile Field Breach: The system calculates that part of the cobot (or an instrument it's holding) is about to move outside the sterile boundary defined for the procedure.

Communication Delay/Glitch: The network latency spikes beyond an acceptable value or control commands stop arriving (which could indicate the surgeon’s console has lost connection).

System Fault: An internal error or a low battery (if running on battery power) occurs, potentially affecting control.

Upon detecting any such anomaly, the system flags it and transitions from passive monitoring to active safety engagement.

Engaging Initial Safety Response (Step 603): Immediately after a hazard is detected, the safety subsystem 170 triggers an automatic response to mitigate risk. Depending on the nature of the issue, this may include:

Deceleration and Pause: The control system 150 is commanded to slow down or halt the motion of arm 110. For example, if a collision risk is detected, the arm might freeze in place or back off slightly from the detected obstacle.

Active Retraction or Evasion: In some cases, the system can intelligently move away from danger. If the arm was moving toward the edge of the sterile field, it can be gently steered back into the allowed zone. If an object is detected in its path, the arm can autonomously retract a few centimeters to maintain a safe distance.

Haptic/Audible Alert to Operator: The system immediately notifies the remote surgeon and any local users of the issue. This can be done by an AR alert (e.g., flashing red overlay or warning icon on AR display 120) and a haptic feedback jolt to the surgeon’s controller, as well as an audible alarm or voice alert (“Obstacle detected” or “Network unstable”) at both ends.

Mode Switch (if needed): The cobot may switch its control mode to a safety mode. For instance, it could enter a gravity-compensation hold, maintaining its current pose stiffly so it doesn’t drift, or a compliance mode where it yields easily if any force is encountered (useful if a person is in contact, to avoid pushing against them).

At this stage, the system essentially puts the surgery in a “safe hold” pattern: the robot ceases aggressive action and maintains or achieves a stable, non-harmful state. It’s important to note that these responses happen very rapidly – within fractions of a second – often before the human operator is even fully aware of the developing hazard.

Assessment and Decision (Step 604): With the immediate risk mitigated by the initial response, the system (and the human operators) assess the situation. The safety subsystem 170 evaluates whether the hazard condition persists or if it was transient. For example, was the obstacle just a momentary intrusion that is now gone, or is someone still in the way? Did the network glitch resolve, or is it ongoing? During this assessment, the system may also run diagnostics – for instance, if a force overload was sensed, the system checks if anything is broken or if forces returned to normal after pausing. The remote surgeon is given information via AR display or console messages about what triggered the safety stop (e.g., “Warning: Non-sterile object detected near surgical field” or “High force detected on instrument tip”). Based on this information, a decision is made on how to proceed.

Recovery or Emergency Override (Step 605): This step represents branching paths depending on the severity of the issue:

If the situation is resolved or deemed safe to continue: The system can resume normal operation in a controlled way. For example, if the obstacle is gone, the surgeon might press a “Continue” confirmation (or give a voice command) once they verify everything is clear, upon which the control system 150 re-engages and the surgery proceeds. The cobot might ramp back up movement gradually to ensure stability. The ML module 160 might also adjust its models if needed (for instance, learning from a false positive if the system was overly cautious).

If the hazardous condition persists or is critical: The system escalates to an emergency override protocol. An emergency override is essentially a full stop and safe shutdown of the active operation to prevent harm. In this case, FIG. 5 shows that the cobot will go into a predefined safe state. This could mean locking the arm in place or moving it to a known safe position (e.g., lifting instruments away from the patient), depending on what is safer in context. It will cut power to certain motors if necessary (to prevent any erratic motion), engage mechanical brakes, or activate redundant safety locks. The system will then await manual intervention. For example, local staff might physically secure the arm or attend to the patient if needed. All the while, the system continues to provide information – e.g., streaming video so the remote surgeon can see what’s happening, and diagnostic data to help troubleshoot.

Alerting and Logging: In an emergency override scenario, the system may also notify hospital systems or other personnel as per safety regulations (for instance, sending a message to an OR control center that an emergency stop occurred at time X). The entire sequence of events is logged in memory for later analysis (to improve the system or for legal documentation, etc.).

Post-Event Analysis and Reset (Step 606): After an emergency override or any significant safety event, the system enters a post-event mode. The cobot 100 will not resume operation until explicitly commanded and until all systems check out. Operators (either the remote surgeon or local staff) perform any needed adjustments – e.g., re-sterilize areas if there was a contamination issue, replace an instrument if it was causing abnormal force, or switch to a backup communication channel if network failure was the culprit. The safety subsystem 170 can run a quick self-test and confirm that sensors are operational and no faults exist. Once everything is cleared, the system can be reset to normal mode and, the surgery can continue (if clinically appropriate), or the procedure can be concluded by other means if necessary. In some designs, the cobot might also have a battery backup allowing it to maintain critical functions (like holding an instrument in place) even if main power is lost, just to safely hand over to a person.

Through the workflow of FIG. 5, it is evident that safety is a paramount aspect of the cobotic interface system. The combination of optical sensors, force sensors, and safety algorithms ensures that the robot behaves as a cautious collaborator. It essentially has a “safety reflex”: much like a human would pull their hand back if they sense danger, the cobot automatically reacts to prevent accidents. This is particularly important in an augmented reality telesurgery scenario, where the surgeon might be immersed in the AR view and could potentially miss something happening peripherally in the real environment – the cobot compensates by being an extra set of eyes and ears on site. Additionally, sterile field enforcement is a unique requirement in surgery that the system addresses: by maintaining virtual boundaries and even using biosensors or machine vision to detect contamination risks, the cobot helps uphold sterility. For example, the system could employ UV or spectral sensors (part of sensor suite 140) to detect biofluids or particles that indicate contamination, and combine that with knowledge of sterile vs. non-sterile zones to alert staff if a contamination event might have occurred.

The emergency override design also ensures that, no matter what, the human operators have final authority. The system is programmed to yield control in any ambiguous or unsafe situation – essentially defaulting to “do no harm.” This means a surgeon can always hit an emergency stop button to instantly immobilize the robot if they feel something is off. Likewise, the system’s automatic override triggers are set conservatively such that it’s more likely to err on the side of stopping than to push limits. While this might occasionally cause interruptions, it guarantees a higher level of safety. As the Aggregated Disclosure emphasizes, the cobot is designed with multiple safety controllers and compliance measures so that it can work collaboratively with humans without causing injury.

In conclusion, FIG. 5’s workflow demonstrates the robust safety net that underlies the cobotic interface for AR-assisted telesurgery. From continuous monitoring and instantaneous hazard response to orderly emergency handling, the system is equipped to maintain patient safety and system integrity even in unexpected circumstances. These safety protocols operate in harmony with the previously described features: for example, the machine learning predictions (FIG. 4) might even foresee certain risks (like the need to avoid a sensitive area) and adjust beforehand, while the control architecture (FIG. 3) provides the hooks for safety overrides at any control level. By incorporating this workflow, the invention ensures that the advancement of telesurgical capabilities through AR and robotics does not compromise on the stringent safety standards required in the medical field.

Overall System Flexibility: It should be noted that the embodiments and workflows described with respect to FIGS. 1–5 are illustrative examples. The invention can be practiced with various modifications in form and detail. Features described in the context of one figure may be combined with features of another, or certain elements may be omitted or substituted, without departing from the scope of the cobotic interface system. For instance, while the figures highlight a humanoid cobot, the safety and AR feedback principles apply equally to non-humanoid configurations as discussed. Likewise, the machine learning assistance can be tailored to different surgical procedures or even other teleoperated tasks beyond surgery, simply by retraining models on the relevant data. The communication interface might employ future high-speed technologies or encryption methods to ensure data integrity and privacy in medical networks. The AR overlay might be implemented as full VR, and references to augmented reality/AR systems herein should be interpreted broadly to include any appropriate extended reality/XR system. In sum, the disclosed system provides a robust platform for augmented reality-assisted remote manipulation, e.g. telesurgery, with an emphasis on real-time responsiveness, intelligent assistance, and safety. The detailed description of the figures above is intended to enable a clear understanding of the best mode of making and using the invention, not to limit the invention’s scope, which is defined by the claims and supported by the full breadth of equivalents and variations that a person of ordinary skill would recognize.

While specific embodiments have been described to enable practice of the disclosed subject matter, they are illustrative and not limiting. Variations, modifications, substitutions, and combinations of the features described can be made by those skilled in the art without departing from the scope of this disclosure. Features described with respect to one embodiment may be used with, or substituted for, features of another. Unless expressly stated otherwise, the terms “comprising,” “including,” and “having” are used in an open, non‑limiting sense; the singular forms “a,” “an,” and “the” include the plural forms; and stated ranges include their endpoints and all subranges. Unless the context clearly indicates otherwise, “and/or” means one or more of the listed items (e.g., “A and/or B” encompasses A alone, B alone, or both A and B); “or” is inclusive (i.e., “A or B” means A, B, or both); and “and” is conjunctive. Headings and ordering are for convenience and do not limit the description. The scope of the claimed subject matter is defined solely by the claims that follow and their legal equivalents.

What is claimed is:

1. A cobotic system for augmented reality-assisted telesurgery operations, comprising:
at least one robotic manipulation platform including at least one robotic arm configured for precise surgical instrument manipulation and one or more sensors providing real-time feedback;
an augmented reality display system configured to present a live surgical video feed with superimposed guidance information to an operator;
a communication interface configured to receive control inputs from a remote operator and transmit real-time video and feedback data to the operator over a secure, low-latency connection; and
a control system operatively coupled to the robotic arm and the communication interface, the control system synchronizing the remote operator’s input with the robotic arm’s movement in substantially real time and providing responsive feedback to the operator.

2. The system of claim 1, wherein the communication interface is configured to provide an encrypted, high-bandwidth link that maintains end-to-end latency below a predetermined threshold to enable near real-time control of the robotic arm.

3. The system of claim 1, wherein the robotic manipulation platform comprises a plurality of multimodal sensors, including an imaging camera, a depth sensor, and force sensors, for capturing intraoperative data and providing corresponding feedback signals.

4. The system of claim 1, further comprising a haptic feedback interface at the remote operator’s station, the haptic feedback interface configured to convey tactile sensations to the remote operator based on forces detected by the robotic arm during instrument manipulation.

5. The system of claim 1, wherein the augmented reality display system is configured to overlay the live surgical video feed with virtual anatomical markers and procedural guidance indicators that assist the operator during the telesurgery.

6. The system of claim 1, wherein the augmented reality display system dynamically updates the overlaid guidance information in response to intraoperative data received from the one or more sensors, thereby reflecting changes in patient anatomy, instrument position, or other surgical conditions in real time.

7. The system of claim 1, further comprising a machine learning module configured to analyze the remote operator’s control inputs and the surgical context to predict the operator’s intended actions and adjust control parameters or system responsiveness proactively.

8. The system of claim 1, wherein the control system includes a feedback loop that continuously synchronizes input commands from the remote operator with movements of the robotic arm with minimal delay, thereby providing substantially instantaneous response to the operator’s actions.

9. The system of claim 1, further comprising safety mechanisms including collision avoidance sensors and control logic to prevent the robotic arm from contacting non-target objects or breaching a predefined safe operating zone, and a sterile-field protection assembly configured to maintain a sterile barrier associated with the surgical site while the robotic arm operates.

10. The system of claim 1, further comprising an automated emergency halt mechanism configured to immediately stop all motion of the robotic arm upon detecting a predefined fault condition or receiving an emergency stop command from the operator.

11. An augmented reality-assisted telesurgery system, comprising:
a surgical robotic apparatus located at a patient site and including at least one robotic arm and a control unit, the robotic arm being configured for precise manipulation of surgical instruments and the control unit being configured to execute control commands;
a remote surgeon console comprising one or more user input control devices and an augmented reality display for presenting a live video feed of the surgical site with superimposed virtual guidance information to a remote surgeon;
a communication network operatively coupling the surgical robotic apparatus and the remote surgeon console, the network enabling secure, bi-directional transmission of control signals and sensor data with low latency for real-time remote operation.

12. The system of claim 11, wherein the augmented reality display presents the live video feed with overlaid anatomical landmarks, recommended instrument pathways, or other procedural guidance cues to assist the remote surgeon during the telesurgery.

13. The system of claim 11, wherein the remote surgeon console further comprises a haptic feedback device configured to apply force or vibrational feedback to the surgeon’s user input control in correspondence with forces sensed by the surgical robotic apparatus at the patient site.

14. The system of claim 11, wherein the communication network is configured as an encrypted, high-speed data link that maintains an end-to-end latency below a predetermined threshold to preserve synchronous operation between the remote surgeon’s inputs and the surgical robotic apparatus.

15. The system of claim 11, further comprising one or more sensors on the surgical robotic apparatus, including an imaging camera and a depth sensor, wherein data from the one or more sensors is used by the augmented reality display to dynamically update the overlaid guidance information in real time during the surgical procedure.

16. The system of claim 11, further comprising a machine learning unit configured to monitor the remote surgeon’s control inputs and surgical workflow, and to anticipate intended surgical actions by the surgeon, adjusting system response or augmenting the displayed guidance information based on the predicted intent.

17. A method for conducting an augmented reality-assisted telesurgery operation, the method comprising:
establishing a secure, low-latency communication link between a surgical robotic apparatus at a patient site and a remote surgeon station;
receiving control commands from the remote surgeon station and conveying the commands to at least one robotic arm of the surgical robotic apparatus;
manipulating one or more surgical instruments with the robotic arm in response to the remote surgeon’s commands;
capturing live video of the surgical site via one or more imaging devices on the surgical robotic apparatus, and transmitting the live video to the remote surgeon station in real time;
overlaying augmented reality guidance information onto the live video feed at the remote surgeon station to provide the remote surgeon with visual indicators aligned with patient anatomy and procedural steps;
providing haptic feedback to the remote surgeon through a force-feedback interface, the haptic feedback being generated based on forces detected at the robotic arm during the instrument manipulation;
dynamically updating the augmented reality overlays during the procedure based on intraoperative data from the surgical robotic apparatus, so that the displayed guidance remains accurate as surgical conditions change;
analyzing the remote surgeon’s control inputs using a machine learning model to predict the surgeon’s intent, and in response preemptively adjusting one or more control parameters or overlay content to enhance system responsiveness;
and automatically executing an emergency stop to halt movement of the robotic arm upon detection of a safety trigger, such as a collision risk or a manual emergency command by the surgeon.

18. The method of claim 17, wherein the step of overlaying augmented reality guidance information includes superimposing visual indicators of anatomical landmarks and recommended surgical instrument paths onto the live video feed to assist the surgeon in navigating the procedure.

19. The method of claim 17, further comprising monitoring the position of the robotic arm relative to a predefined safe operating zone during the telesurgery and automatically restricting or modifying incoming control commands to prevent the robotic arm from colliding with patient tissues or surgical equipment outside of an intended operative area.

20. The method of claim 17, wherein the step of analyzing the remote surgeon’s control inputs using the machine learning model includes identifying patterns in the surgeon’s input movements and anticipating subsequent intended actions, such that the system preemptively adjusts control responsiveness or updates the augmented reality overlays based on the anticipated actions.
